(19) United States
(12) Patent Application Publication
(10) Pub. No.: US 2009/0047258 A1
(43) Pub. Date: Feb. 19, 2009

(54) COMPOSITIONS AND METHODS FOR REVERSING AGE-RELATED CHANGES IN EXTRACELLULAR MATRIX PROTEINS

(75) Inventors: Lucian V. Del Priore, Tarrytown, NY (US); Tongalp Tezel, Louisville, KY (US); Henry J. Kaplan, Louisville, KY (US)

Correspondence Address: WilmerHale/Columbia University, 399 PARK AVENUE, NEW YORK, NY, 10022, US

(73) Assignee: THE TRUSTEES OF COLUMBIA UNIVERSITY IN THE CITY OF NEW YORK, New York, NY (US)

(21) Appl. No.: 11/659,151

(22) PCT Filed: Aug. 2, 2005
(86) PCT No.: PCT/US2005/027545

§ 371 Date: Sep. 16, 2008

Related U.S. Application Data
(60) Provisional application No. 60/598,858, filed on Aug. 2, 2004.
(60) Provisional application No. 60/653,457, filed on Feb. 16, 2005.

(51) Int. Cl. A61K 35/12; A61K 38/16; A61P 17/00; A61P 27/02; A61P 25/00

(52) U.S. Cl. 424/93.7; 514/12

(57) ABSTRACT

The invention is directed to compositions and methods for reversing age-related changes in extracellular matrix proteins, particularly the collagen framework of basement membranes of various organs. The invention has particular application to age-related changes that impair Retinal Pigment Epithelium (RPE) cell repopulation of human Bruch's membrane.

### CROSS REFERENCE TO RELATED APPLICATIONS

This application claims priority to U.S. Provisional Application Ser. No. 60/598,858, filed Aug. 2, 2004, and to U.S. Provisional Application Ser. No. 60/653,457, filed Feb. 16, 2005.

### FIELD OF THE INVENTION

The invention is directed to compositions and methods for reversing age-related changes in extracellular matrix proteins, particularly the collagen framework of basement membranes of various organs. The invention has particular application to age-related changes that impair Retinal Pigment Epithelium (RPE) cell repopulation of human Bruch's membrane.

### BACKGROUND OF THE INVENTION

Collagen protein serves as a key structural component of connective tissues such as, for example, skin and ligaments. Collagen fibers form a supporting network responsible for mechanical characteristics such as strength, texture, and resilience of connective tissues. Like all material, collagen is subject to wear and tear: it slowly breaks down over time during the aging process. Collagen breakdown results in a number of diseases and biological disorders.

For example, changes in the inner collagen layer (“ICL”) and basement membrane of the retina can cause age-related macular degeneration (“AMD”), a leading cause of blindness in the elderly. Earlier studies showed that age-related changes in the ICL and other layers of human Bruch's membrane inhibit retinal pigment epithelium (“RPE”) cell repopulation of human Bruch's membrane, and these steps may be involved in the pathogenesis of AMD and may inhibit RPE repopulation of human Bruch's membrane by transplanted RPE.

Currently, photodynamic therapy, thermal laser photocoagulation, and intravitreal injection of anti-VEGF drugs are the only clinically validated treatments for selected cases of exudative AMD. Thermal laser treatment coagulates new choroidal vessels at the cost of destroying the overlying sensory retina and creating an absolute central scotoma. Even so, only less than 20% of patients with exudative AMD are eligible for laser photocoagulation, and half of them experience persistent or laser photocoagulation. Photodynamic therapy reduces the rate of visual loss due to well-defined choroidal neovasucularization but does not lead to significant visual improvement in most individuals.

These limitations have led to the development of alternative treatment modalities, such as systemic interferon, radiotherapy, subfoveal membranectomy, macular translocation, and anti-angiogenic pharmacological agents, such as anti-VEGF antibody, anti-VEGF aptamer, triamcinolone, and anecortave acetate. These techniques all aim to obliterate new choroidal vessels, to decrease plasma leakage from such vessels, or both. The techniques often require multiple treatment sessions, they rarely improve central vision, and at best they merely retard visual deterioration. Eventually, persistent exudation from the subretinal fibrovascular tissue causes fibrovascular scar formation and continuing disruption of the relationship among the choriocapillaris, RPE, and photoreceptors. Ultimately, photoreceptor cell death and loss of central vision result.

Unfortunately, simple excision of the subfoveal neovascular membrane in AMD leaves a large RPE defect under the fovea, due to the removal of native RPE along with the surgically removed neovascular complex. Resultant persistent RPE defects in AMD lead to the development of progressive choriocapillaris and photoreceptor atrophy.

Attempts to repopulate Bruch's membrane defects with native or transplanted RPE cells have not been successful. Histopathology after adult human RPE transplantation in one eye with AMD showed failure of transplanted RPE cells to attach and form a complete monolayer under the fovea. RPE cells must reattach to a substrate to avoid apoptosis. We have shown previously that the age of the Bruch's membrane and the layer of Bruch's membrane available for cell attachment have a profound effect on the fate of human RPE seeded onto Bruch's membrane in vitro.

Cellular replacement in AMD remains a plausible clinical goal. The neurosensory retina overlying choroidal neovascularization has the potential to recover, as evidenced by the preservation of foveal photoreceptors in eyes with exudative AMD. Even at later stages of AMD, 25% to 30% of the photoreceptors have been shown to remain structurally intact. Foveal translocation surgery has also supplied clues that the remaining photoreceptors are sufficient to restore central vision, as some patients achieve a final visual acuity of 20/40 or better once the photoreceptor-RPE interface is restored.

It is known that adult human RPE cells can attach to and repopulate the innermost basal laminar layer of both young and aged (<60 years) Bruch's membrane, although they fail to survive on the deeper layers of aged human Bruch's membrane. Thus, the fate of an RPE graft at the time of surgical excision of a neovascular complex is dependent on the layer of Bruch's membrane exposed, as well as the presence of disease (AMD) within Bruch's membrane. However, Bruch's membrane may be abnormal in patients with AMD: thickening of Bruch's membrane and the formation of basal laminar deposits, basal linear deposits, and drusen occur early in the pathogenesis of AMD. Furthermore, surgical removal of subfoveal choroidal neovascularization in AMD may disrupt the inner layers of Bruch's membrane, so that the lamellae of Bruch's membrane available for RPE reattachment may not be uniform throughout the transplantation bed. RPE cells plated onto deeper layers of aged human Bruch's membrane fail to attach and eventually die by apoptosis. Failure of RPE to survive and repopulate diseased and damaged areas of Bruch's membrane may be one of several factors accounting for the fact that an uncontrolled series of human transplantation studies with allogenic and autologous fetal and adult human RPE failed to show any biological benefit. This is in contrast to the anatomic and functional success of RPE transplantation in animal models that lack age-related ultrastructural alterations in human Bruch's membrane. Therefore, restoration of foveal vision in exudative AMD may require modification of aged Bruch's membrane to improve RPE repopulation of this structure.

It has been shown previously that age-related alterations in the molecular composition and ultrastructure of human Bruch's membrane make it an unfavorable substrate for the attachment and survival of grafted RPE cells. Apoptotic mechanisms are activated within the harvested RPE graft as soon as cells are detached from their native substrate during the harvesting procedure. RPE cell death can be suppressed by RPE reattachment and subsequent spreading on a substrate through the interaction between integrin receptors on the basal surface of RPE and their specific ligands within the ECM. Failure to reestablish this interaction after RPE harvesting inevitably results in rapid RPE death by apoptosis. The invention shows that age-related structural alterations in human Bruch's membrane can be completely or partially reversed by cleaning and coating Bruch's membrane and coating it with ECM proteins. Such treatment can reestablish the native ECM framework to an extent adequate enough to alter the dismal fate of human RPE grafts seeded onto the ICL of aged Bruch's membrane.

Previous studies have demonstrated that several structural and molecular alterations occur within human Bruch's membrane as a function of age. These changes, which disrupt the delicate molecular architecture of Bruch's membrane, include (1) structural changes in the main collagen framework, including cross-linking and deposition of long-spaced collagen; (2) qualitative and quantitative changes in the native ECM molecules; (3) deposition of abnormal extrinsic molecules; and (4) macromolecular changes in the structure of Bruch's membrane, such as drusen formation, calcifications, and cracks or loss of inner layers due to inadequate basal membrane regeneration, as in geographic atrophy. However, studies of AMD have been hampered by the lack of useful animal models, in part because animals do not get AMD.

In summary, it was previously demonstrated that age-related changes within the ICL and other layers of Bruch's membrane inhibit RPE cell repopulation after subfoveal membranectomy in AMD. There exists a need to develop a process and a composition for enhancing the reattachment and repopulation of human RPE seeded on denuded, aged human Bruch's membrane. The present invention demonstrates the reversal of these age-related changes by reengineering Bruch's membrane—namely, by cleaning the ICL with a nonionic detergent and refurbishing it with ECM proteins (laminin, vitronectin, fibronectin).

Furthermore, fibroblasts in skin cells produce collagen, and when needed, fibroblasts replace broken collagen fibers with new ones. The ability of the skin to replace damaged collagen diminishes with age. Consequently, more gaps and irregularities develop in the collagen mesh-work. This process eventually leads to wrinkles. Thus, there is also a need to develop a process and a composition for preventing and eliminating wrinkles and other age-related changes within human skin.

Additionally, age-related changes in collagen mesh also occur in other organs and tissues such as blood vessels and internal organs. Just as age-related changes (such as collagen cross-linking, elastin fragmentation, and deposits of abnormal material) in Bruch's membrane, may precede cellular changes by decades, similar age-related changes occur in the ECMs of various other tissues and organs. For example, skin wrinkling is characterized by collagen cross-linking, fragmentation of elastin, and alteration of matrix metalloproteinase activity. In Alzheimer's disease, there is an aggregation of β-amyloid within the ECM of the brain that induces secondary changes in neural cells. Aging of the ECM is also responsible for a pro-oncogenic milieu that manifests itself as an exponentially increasing incidence of epithelial cancers with aging.” In the eye, age-related changes in the glycosaminoglycan in the trabecular meshwork contribute to the development of open-angle glaucoma.

Therefore, there is also a need to reverse age-related changes in the extracellular matrices, including but not limited to the collagen meshwork in other organs and tissues.

### SUMMARY OF THE INVENTION

The invention solves the problem of deterioration of basement membranes and related cellular dysfunctions.

The invention provides compositions and methods for reversing deterioration, such as age-related changes, within the extracellular matrix proteins or collagen framework of the basement membranes of various mammalian tissues and organs. The invention provides for repair of such matrix proteins and collagen framework. In some embodiments, the invention reverses age-related changes in fibrillar collagen proteins. In other embodiments, the invention reverses age-related changes in non-fibrillar collagen proteins. In yet other embodiments, the invention provides methods of treatment and prevention of such ailments as age-related macular degeneration, open-angle glaucoma, skin wrinkles, Alzheimer's disease, and a host of other maladies caused by the deterioration of matrix protein within membranes.

### DESCRIPTION OF FIGURES

FIG. 1 demonstrates reattachment ratios of RPE on modified aged-ICL. In some embodiments, ECM protein coating (ECM-P) increased the reattachment ratios of both RPE cell lines to aged ICL, whereas the cleaning process (0.2% Triton X-100 in 0.2 M sodium citrate solution at 4 degrees C. for 4 minutes) decreased the reattachment ratio. However, ECM protein coating after the cleaning procedure restored the reattachment ratio of fetal RPE cells, but it failed to show a similar effect on ARPE-19 cells.

FIG. 2 demonstrates apoptosis ratios of RPE on modified aged ICL. In some embodiments, the highest apoptosis ratios were observed on untreated ICL. The apoptosis rate of fetal RPE was higher than ARPE-19 cells on untreated ICL. ECM protein coating (ECM-P) lowered the apoptosis ratio significantly in both cell types. Cleaning or cleaning and coating further decreased the apoptosis ratios of fetal RPE cells, but did not alter the apoptosis rate of ARPE-19 cells.

FIG. 3 demonstrates proliferation ratios of RPE on modified aged ICL. Twenty-four hours after growth induction there was no significant proliferation on untreated and ECM-protein-coated (ECM-P) ICI, whereas RPE cells plated on cleaned or cleaned and ECM-protein-coated ICI proliferated. The shaded area indicates failure of RPE proliferation. FIG. 4 demonstrates surface coverage of RPE on modified aged ICL 17 days after plating. In some embodiments, few cells survived on untreated and ECM-coated (ECN-P) ICL. Although the initial attachment ratios were low on cleaned ICL, RPE cells proliferated on this layer, resulting in higher surface coverage. The highest surface coverage was obtained with ARPE-19 cells on cleaned and ECM-protein-coated ICL.

FIG. 5 shows scanning electron microscopy (SEM) of ICL from an 84-year-old donor. (A) Untreated ICL revealed cross-linked bundles of collagen (arrows). Small globular structures on the collagen fibers probably represent aggregates of abnormal ECM proteins (arrowheads). Macrodeposits filled interfibrillar spaces (*). (B) ECM protein coating without cleaning increased the amount of ECM protein aggregates on the collagen matrix (arrowhead). Cross-linking of collagen fibers was not affected by the coating (arrows). (C) Cleaning with Triton X-100 and sodium citrate removed the aggregates of ECM proteins and debris and resulted in gaps between collagen fibers (*). Note that cross-links between collagen fibers were broken, yielding individual fibers (arrowheads). In some areas there was incomplete separation of the macrofibers (arrows). (D) Cleaning and subsequent ECM protein coating broke the cross-links between collagen fibers and resulted in deposition of ECM proteins onto the regenerated collagen framework (Arrowheads). The ECM proteins coating the surface were smaller in size and did not form multimeric aggregates on the native matrix. The gaps (arrows) remained between the collagen fibers, probably corresponding to areas where macrodebris was removed (compare to A). Bars, 0.5 μm.

FIG. 6 shows transmission electron microscopy (TEM) of ICL from an 84-year old donor. (A) Untreated ICL composed of bundles of cross-linked collagen (arrows) accompanied by numerous electron-lucent deposits (arrowheads). (B) ECM protein coating increased the number of electron-dense particles (arrowheads) which formed aggregates among the cross-linked collagen bundles (arrows). (C) Cleaning broke the cross-links between collagen fibers (arrows) with resulting gaps within the collagen matrix (asterisk). An electron-dense halo surrounds the residual deep electron-lucent lipoproteinaceous debris, possibly representing exposure of the hydrophilic tails of the proteins after detergent treatment (arrowheads). (D) Combined cleaning and coating resulted in breakdown of the cross-links between collagen fibers and an increase in electron-dense particles that were evenly distributed within the collagen framework and did not form aggregates (arrowheads). Bars: (A, B) 1 μm; (C, D) 0.5 μm.

FIG. 7 shows SEM of RPE on ICL. (A) RPE cells plated on untreated ICL failed to flatten within 24 hours. (B) ECM protein coating increased the number of attached cells; however, these cells remain rounded. (C) Cleaning of the ICL resulted in fewer attached RPE cells but they were flattened. (D) Combined cleaning and ECM protein coating yielded flattened RPE cells with differentiated cell features such as microvilli. RPE cells extended foot processes and formed focal adhesion plaques (arrows). Bars: (A) 7.5 μm; (B, C) 5.0 μm; (D) 3.0 μm.

FIG. 8 shows SEM of RPE 17 days after plating. (A) Occasional cellular debris was noted on untreated aged ICL. (B) ECM protein coating did not increase RPE cell survival. Seventeen days after plating, surface showed numerous areas of cellular debris. (C) Surviving RPE on cleaned ICL showed long cellular extensions (arrows). (D) RPE cells plated on cleaned and ECM-protein-coated ICL proliferated and formed patches of an epithelial monolayer (arrows). Bars: (A) 5 μm; (B) 30 μm; (C) 25 μm; (D) 50 μm.

FIG. 9 demonstrates the effects of coating and/or cleaning of the ICL on RPE reattachment, apoptosis, and surface repopulation. Column 1: young Bruch's membrane contains normal collagen with globular ECM proteins (grey ellipsoids). Changes develop within Burch's membrane as a function of age, including collagen cross-linking (shown as squiggled lines between collagen fibers) and deposition of abnormal material (polygons with light shading; row 2). Coating alone did not remove the abnormal structures but simply increased the number of ligands available for cell attachment (row 3). Cleaning with Triton X-100 and sodium citrate removed the abnormal deposits and cross-links but also removed most of the ECM proteins (row 4). The combination of cleaning plus coating restored Bruch's membrane ICL to its normal morphology (row 5). Column 2: RPE cell attachment was higher on young and intact Bruch's membrane (row 1) than on older ICL (row 2). RPE attachment was higher after the surface was coated with ECM proteins without cleaning (row 3). The attachment rate was lowered by cleaning the surface, which removed the abnormal deposits and normal ECM molecules (row 4). The attachment rate was highest after the surface was cleaned and then coated with new ECM proteins (row 5). Column 3: post—attachment behavior of RPE. RPE cells flatten and undergo proliferation on young Bruch's membrane. However, on uncoated ICL from older individuals, RPE cells failed to flatten and proliferate, and they had the highest apoptosis rate (row 2). Coating the surface with ECM proteins decreased the apoptosis ratio (row 3), but attached cells did not flatten on the surface and failed to proliferate. Cleaning alone decreased the attachment ratio significantly; however, attached cell proliferated (row 4). Cleaning of the surface followed by coating with ECM proteins resulted in RPE flattening and proliferation and the lowest RPE apoptosis rate (row 5). Column 4: fate of the seeded RPE. RPE cells easily resurfaced the young and intact Bruch's membrane (row 1), whereas on old ICL, they were all eliminated ultimately by apoptosis (row 2). ECM protein coating increased RPE cell attachment but did not allow the cells to resurface the old ICL (row 3). Cleaning alone resulted in some surface coverage at 17 days (row 4), and this effect was maximized by cleaning followed by recoating (row 5).

### DETAILED DESCRIPTION OF THE INVENTION

As noted above, this invention provides compositions and methods for reversing age-related changes within the extracellular matrix proteins or collagen framework of the basement membranes of various mammalian tissues and organs.

In one embodiment, the invention provides a composition and a method that reverses the age-related changes in collagen proteins that impair Retinal Pigment Epithelium (RPE) cell repopulation of human Bruch's membrane. The method of the invention utilizes at least two steps: (1) cleaning of native Bruch's membrane followed by treatment with a weak acid such as sodium citrate; and (2) refurbishing of Bruch's membrane by coating it with extracellular matrix protein (ECM) molecules such as laminin, vitronectin, and fibronectin. The cleaning step may be performed with i) a non-ionic detergent such as Triton-X 100, ii) a biodetergent such as a fluorinated surfactant, or iii) a lipid-degrading enzyme, such as lipoprotein lipase.

In some more specific embodiments, the invention provides methods which include breaking the malformed or otherwise cross-linked collagen in the ICL with known collagen-crosslink-breakers, such as N-phenyl thiazolium bromide. Such treatment increases the reattachment and survival of RPE cells on aged human ICL. The use of this treatment is thought to facilitate resurfacing of human Bruch's membrane after submacular surgery in AMD.

More specifically, in one embodiment, this invention provides a method of reversing age-related changes in extracellular matrix proteins such as collagen, comprising the steps of (i.) cleaning a membrane made up of collagen protein with a composition comprising (a.) a non-ionic detergent; and (b.) a weak acid; and (ii.) coating that membrane with extracellular matrix protein.

In a more specific embodiment, the invention provides a method wherein the extracellular matrix protein used for coating is a composition comprising at least two of laminin, fibronectin, and vitronectin.

In another more specific embodiment, the invention provides a method wherein the extracellular matrix protein used for coating is a composition comprising laminin, fibronectin, and vitronectin.

In a more specific embodiment, the invention provides a method wherein the extracellular matrix protein composition contains from about 300 to about 400 micrograms/ml of laminin; from about 200 to about 300 micrograms/ml of fibronectin; and from about 30 to about 40 micrograms/ml of vitronectin.

In a more specific embodiment, the invention provides a method wherein the extracellular matrix protein composition contains from about 300 to about 350 micrograms/ml of laminin; from about 220 to about 280 micrograms/ml of fibronectin; and from about 30 to about 40 micrograms/ml of vitronectin.

In another embodiment, this invention provides the method above, with the additional step of breaking down collagen cross-links with N-phenyl thiazolium bromide or with another agent capable of breaking down collagen cross-links to restore the native three-dimensional matrix structure.

In a still more specific embodiment, the non-ionic detergent is Triton-X 100. In another more specific embodiment, the weak acid is an organic acid whose pKa is between about 3 and about 6. In another more specific embodiment, the weak acid is an organic acid whose pKa is between about 3 and about 5.

In another more specific embodiment, the weak acid is sodium citrate.

In another embodiment, the invention provides compositions and methods that reverse the age-related changes in the collagen framework in skin, and thereby prevents or eliminates wrinkles.

In another embodiment, the invention provides compositions and methods that reverse the age-related changes in β-amyloid protein.

In another embodiment, the invention provides compositions and methods that reverse age-related changes in the collagen network in other organs and tissues such as blood vessels, internal organs, and other tissues.

In another embodiment, this invention provides a method to facilitate transplantation of RPE cells, including in particular fetal RPE cells and ARPE-19 cells.

In another embodiment, this invention provides a method to facilitate grafting of cells onto Bruch's membrane.

In some embodiments of the invention, a simple cleaning of the aged ICL lowers the reattachment of both RPE cell types, possibly by removal of ECM proteins serving as adhesion molecules. This conclusion is supported by the disappearance of globular proteins from and between collagen fivers detected by SEM. This conclusion is further supported by an observation of the invention that replenishing the ECM proteins—namely, laminin, vitronectin, and fibronectin—significantly increases the attachment rate of fetal RPE on cleaned ICL. Failure to restore the reattachment of ARPE-19 cells by ECM protein coating of cleaned ICL suggests these cells may depend on different ECM receptors or on a unique three-dimensional architecture of binding sites for attachment. However, different cell lines—and even different passages of the same cell line—may express different integrin heterodimers to attach to a substrate.

Cleaning alone may decrease the reattachment of both RPE cell lines. Supplying the ICL with high concentrations of ECM proteins alone increases RPE reattachment but does not result in RPE flattening. A random polymerization pattern of supplementary ECM proteins on the ICL may prevent them from acquiring their native three-dimensional organization and expose their binding epitopes at regular intervals. Addition of ECM proteins on the ICL increases the RPE attachment rate, but it does not decrease the apoptosis rate. This suggests that increased RPE attachment alone is not sufficient to increase survival on aged ICL.

On the appropriate substrate, RPE cell adhesion to a surface is followed by cell spreading, formation of focal adhesions, and the development of stress fibers with subsequent cell proliferation and migration. Cell proliferation is controlled by many of the same signaling proteins that play a role in adhesion, and also requires a proper interaction of integrin receptors with their ECM ligands. In some embodiments, RPE cells attached to cleaned ICL flatten and proliferate. However, on cleaned and ECM-protein coated ICL, where RPE obtain the highest proliferation rate, the inventors were able to populate approximately one third of the bare ICL during the observation period. Further modification of the ICL or an increase in the number of RPE cells may allow complete resurfacing of the epithelial defect.

The fate of the human fetal and ARPE-19 cell lines seeded onto untreated aged human ICL is similar to that of adult human RPE cells, although ARPE-19 cells are more resistant to detachment-induced apoptosis. The resistance of ARPE-19 cells to apoptosis on untreated ICL may be due to a deficiency in two major apoptosis execution pathways with the induction of nuclear calcium-dependent endonucleases activation of the interleukin-1β-converting enzyme family of proteases. Despite their increased resistance, alterations in the chemical composition of the aged ICL can still induce apoptosis in ARPE-19 cells and ultimately lead to the same fate as that of adult and fetal human RPE cells.

Structural alterations can be induced by submacular surgery, because excised neovascular membranes in AMD eyes contain fragments of the basal lamina and deeper layers of Bruch's membrane, thus exposing the ICL and perhaps other layers. The chemical treatments described herein, which are used to reengineer the aged human Bruch's membrane may act by (1) liquefying and extracting membranous lipoprotein debris from the ICL to expose ECM protein receptors on native collagen fibers; (2) 14/32 reestablishing the native collagen framework by dissolving long-spacing collagen and breaking collagen cross-links; and (3) polymerizing a layer of ECM proteins on to the rejuvenated core collagen matrix of Bruch's membrane. A nonionic detergent, for example, Triton X-100, extracts membranous debris from the aged Bruch's membrane while preserving the anionic glycosaminoglycan bridges between the collagen fibrils and the native structure of collagen. At the concentrations used, Triton X-100 dissolves the membranous debris of age-related photoreceptor outer segments without disrupting the ultrastructure of the matrix. At these concentrations, Triton X-100 apparently does not interfere with the subsequent adhesion of ECM proteins to the collagen fibers and apparently allows them to polymerize in their native form on the collagen matrix. Detergent treatment before ECM protein coating avoids the binding of ECM molecules to lipoprotein debris with a consequent abnormal configuration. The reducing agent sodium citrate solubilizes the lipid debris and facilitates the breakdown of age-related pentosidine cross-links between collagen fibers.

In theory, the removal of the lipoprotein debris and the secondary increase in anionic binding sites may induce a shift toward hydrophilicity and increased hydraulic conductivity of the ICL. Chemical treatments used to reengineer the aged human Bruch's membrane appear to act by liquefying and extracting membranous lipoprotein debris from the ICL to expose ECM protein receptors on native collagen fibers, thus reestablishing the native collagen framework by dissolving long-spacing collagen and breaking collagen cross-links. This framework, in turn, allows proteins subsequently placed on this surface to polymerize onto the rejuvenated core collagen matrix of Bruch's membrane.

The present invention demonstrates reversing the age-related changes in Bruch's membrane composition and structure in AMD. A correction of this change leads to changes in RPE behavior. This view offers a different perspective of the role of the age-related changes in the ECM in regulating cell behavior and presents a unique opportunity to intervene by reversing this process.

The precise quantities of anionic detergent, of N-phenyl thiazolium bromide (or other agent for breaking down cross-linked collagen), and of extracellular matrix protein employed in therapeutic procedures will depend on the disease, and on the condition of the particular patient. The present invention is explained in greater detail in the following non-limiting examples.

### EXAMPLES

### Example 1

### Preparation of RPE Cultures

All study protocols adhered to the provisions of the Declaration of Helsinki for research involving human tissue. Human fetal RPE cells were harvested from 14- and I7-week-old human fetuses processed within 6 hours. The techniques for harvesting and culturing the RPE cells, known to a person skilled in the art, were used. Briefly, on receipt, eyes were cleaned of extracellular tissue. A circumferential scleral incision was made 1.5 mm posterior to the limbus, and the sclera was peeled away. The eyecup was then incubated with 25 U/mL dispase (Invitrogen-Gibco, Grand Island, N.Y.) for 30 minutes and rinsed with CO₂-free medium (Gibco). Loosened RPE sheets were collected with a Pasteur pipette and plated onto bovine corneal endothelium-ECM-coated, 60-mm treated plastic dishes (Falcon; BD Biosciences UK, Plymouth, UK). The cells were incubated in a humidified atmosphere of 5% CO₂ and 95% air at 37° C. and maintained in Dulbecco's modified Eagle's medium (DMEM H16; Invitrogen-Gibco) supplemented with 15% FBS, 100 IU/mL penicillin G, 100 μg/mL streptomycin, 5 μg/mL gentamicin, 2.5 μg/mL amphotericin B, and 1 ng/mL recombinant human basic fibroblast growth factor (bFGF; Invitrogen-Gibco), to promote RPE cell growth. The medium was changed every other day and the cells observed daily. Cells became confluent in approximately 10 days, and confluent cultures were passaged by trypsinization. First-passage RPE cell lines were used in these experiments.

The ARPE 19 cell line was obtained from the American Type Culture Collection (Manassas, Va.). This is a line of spontaneously immortalized RPE cells that have morphologic and functional characteristics similar to those of adult human RPE cells. Cells were maintained in a 1:1 mixture of DMEM and Ham's F-12 with HEPES buffer containing 20% FBS (Invitrogen-Gibco), 56 mM final concentration sodium bicarbonate, and 2 mM L-glutamine (Sigma-Aldrich, St. Louis, Mo.) and incubated at 37° C. in 10% CO₂.

### Example 2

### Cytokeratin Labeling

Cells were stained with a pancytokeratin antibody to verify that all cells were of epithelial origin. For this purpose, harvested RPE sheets were rinsed in phosphate-buffered saline (PBS), fixed with 4% paraformaldehyde for 30 minutes, and washed again with PBS. The cells were treated for 1 hour at room temperature with 3% bovine serum albumin (Sigma-Aldrich) in PBS to block nonspecific binding sites. The cells were then incubated at 37° C. for 1 hour with an FITC-conjugated monoclonal anti-pan cytokeratin antibody to cytokeratin −5, −6, and −8 (Sigma-Aldrich). The cells were washed three times with PBS and examined under a fluorescent microscope. An irrelevant isotypic IgG primary antibody (anti-human von Willebrand antibody; Sigma-Aldrich), coupled with an FITC-conjugated secondary antibody was also used and showed no background staining. All the harvested cells were positive for pancytokeratin, indicating that the cells were of epithelial origin.

### Example 3

### Harvesting of Human Bruch's Membrane Explants

Explants of the inner collagen layer (ICL) of human Bruch's membrane were prepared from the peripheral retinas of eyes of four elderly donors (average age, 77±6 years [SD]; range, 69-84 years old) obtained within 24 hours of death. The harvesting technique known to a person skilled in the art has been used. Briefly, a full-thickness circumferential incision was made posterior to the ora serrata, and the anterior segment and vitreous were carefully removed. The posterior pole of each eyecup was inspected visually with direct and retroillumination under a dissecting microscope, and globes were discarded if there was any evidence of sub-retinal blood, previous surgery, or any extensive structural or vascular alteration of the posterior segment due to a disease process, such as proliferative diabetic retinopathy or proliferative vitreoretinopathy. The eyecups were put in CO₂-free medium (Invitrogen-Gibco), and a scleral incision was made 3 mm from the limbus and extended 360°. Four radial incisions were then made, and the sclera was peeled away. A circumferential incision was made into the subretinal space 1 mm posterior to the ora serrata. The choroid-Bruch's membrane-RPE complex was then carefully peeled toward the optic disc and removed after its attachment to the optic nerve was trimmed. Native RPE cells were removed by bathing the explant with 0.02 N ammonium hydroxide in a 50-mm polystyrene petri dish (Falcon; BD Biosciences) for 20 minutes at room temperature, followed by washing three times in phosphate-buffered saline (PBS). The Bruch's membrane explant from the fellow eye was prepared by removing the RPE with 0.02 N ammonium hydroxide as just described. The Bruch's membrane explant was then floated in complete Freund's medium (CFM) over a 12- to 18-μm-thick hydrophilic polycarbonate-polyvinyl pyrrolidone membrane with 0.4-μm pores (Millipore, Bedford, Mass.) with the basal lamina facing the membrane. The curled edges were flattened from the choroidal side with fine forceps without touching Bruch's membrane. Four percent agarose (Sigma-Aldrich) was poured on the Bruch's membrane-choroid complex from the choroidal side, and the tissue was kept at 4° C. for 2 to 3 minutes to solidify the agarose. The hydrophilic membrane was peeled off along with the basal lamina of the RPE, thus exposing the bare ICL. Circular buttons (6-mm diameter) were then trephined from the peripheral Bruch's membrane on a Teflon sheet and placed on 4% agarose at 37° C. in non-treated polystyrene wells of a 96-well plate (Corning Costar Corp., Cambridge, Mass.). The agarose solidified within 2 to 3 minutes at room temperature, thus stabilizing the Bruch's membrane explant. The wells were gently rinsed with PBS three times for 5 minutes, gamma sterilized (20,000 rad), and then stored at 4° C.

### Example 4

### Various Treatments of ICL of Bruch's Membrane

Explants containing ICL of aged Bruch's membrane on the apical surface were prepared as described earlier and processed further to create four experimental plating surfaces:

(1) cleaned ICL: For this purpose, triplicate explants were treated with 0.1% Triton X-100/0.1% sodium citrate solution for 20 minutes at 4° C.;

(2) ECM-protein-coated ICL: To coat ICL with ECM protein, another set of triplicate buttons were incubated with an ECM protein mixture containing laminin (330 μg/mL), fibronectin (250 μg/mL), and vitronectin (33 μg/mL) at 37° C. for 30 minutes;

(3) cleaned and ECM-protein-coated ICL: Some buttons were first cleaned and then coated with ECM protein; and

(4) untreated buttons: These were used to determine the fate of the fetal and ARPE-19 RPE cell lines on aged ICL.

After the cleaning and/or coating process, the exposed surfaces were washed three times with PBS for 5 minutes, and explants were stored at 4° C.

### Example 5

### RPE Reattachment Studies

Confluent cell cultures were synchronized by placing them in phenol-free MEM (Invitrogen-Gibco) without scrum for 24 hours before harvesting with 0.25% trypsin/0.25% EDTA in Hanks' balanced salt solution for 10 minutes. Two milliliters of 0.1 mg/mL aprotinin (Sigma-Aldrich) in HEPES buffer (pH 7.5) was added to quench the trypsin reaction, and the cell suspension was centrifuged for 5 minutes at 800 rpm. The cell pellet was washed three times and then re-suspended in phenol red-free MEM without serum. The number of cells was determined by cell counter (model Z-1; Coulter Scientific, Hialeah, Fla.), and cell viability was assessed with a kit (Live/Dead Viability Kit; Molecular Probes, Eugene, Oreg.). At least 250 cells were examined under 100× magnification, and the viability was expressed as the average ratio of live cells to the total number of cells in these three different areas.

Fifteen thousand viable RPE cells were plated on different layers of Brach's membrane explants and serum- and phenol-free MEM containing 100 IU/mL penicillin G, 100 μg/mL streptomycin, 5 μg/mL gentamicin, and 2.5 μg/mL amphotericin B was added to reach a final volume of 200 μl in each well. At this plating density, the RPE cells covered approximately 15% of the plating area, assuming a cell diameter of 20 μm. RPE cells were allowed to attach to the surface for 24 hours in a humidified atmosphere of 95% air/5% CO₂ at 37° C. in phenol red-free MEM (Invitrogen-Gibco) without serum. Unattached cells were removed from the tissue culture plates by gently washing the wells three times with MEM.

### Example 6

### Assay for RPE Adhesion

The number of attached live RPE cells in each well was determined with a calorimetric assay that indirectly estimates the number of live cells by measuring intracellular dehydrogenase activity (CellTiter 96 Aqueous non-radioactive cell proliferation assay; Promega, Madison, Wis.). Dehydrogenase enzymes found in live cells reduce MTS (3-(4,5-dimethylthiazol-2-yl)-5-(3-carboxymethoxyphenyl)-2-(4-sulfophenyl)-21-tetrazolium) into the water-soluble formazan in the presence of an electron-coupling agent (phenazine methosulfate, “PMS”). The quantity of the formazan product can be determined from the absorbance at 490 nm and is directly proportional to the number of living cells in culture.

The assay was performed in dark conditions, because of the light sensitivity of MTS and PMS. MEM (100 μL) without phenol red was added to each well. The added solution contained 1.0 g/mL glucose in a bicarbonate-based buffer that maintains the pH at 7.3 to 7.4 in 5% CO₂ and 95% air, thus minimizing the effects of changes in glucose and pH on the colorimetric assay. Twenty μL of freshly prepared MTS/PMS solution (20:1) was added to each well, resulting in a final concentration of 333 μg/mL MTS and 25 μM PMS. Plates were incubated for 4 hours at 37° C., and 100 μL of medium from each well was transferred to a corresponding well of another 96-well plate and read at 490 μm with an ELISA plate reader. The corrected absorbance was obtained by subtracting the average optical density reading from triplicate sets of controls containing the Bruch's membrane explant on 4% agarose without plated cells. The number of viable cells was estimated from standardized curves obtained by plating 100 to 13,000 viable, synchronized fetal RPE and ARPE-19 cells separately in triplicates on Bruch's membrane explants stabilized on 4% agarose. A linear relationship (r=0.93, 0.96 for fetal RPE and ARPE-19 cells, respectively) was observed between the number of viable cells and the absorbance at 490 nm (data not shown). The RPE reattachment ratio for a substrate was the ratio of attached cells to the entire plated cell population for that substrate: ratio=[attached/(attached+unattached)].

### Example 7

### Reattachment Ratio

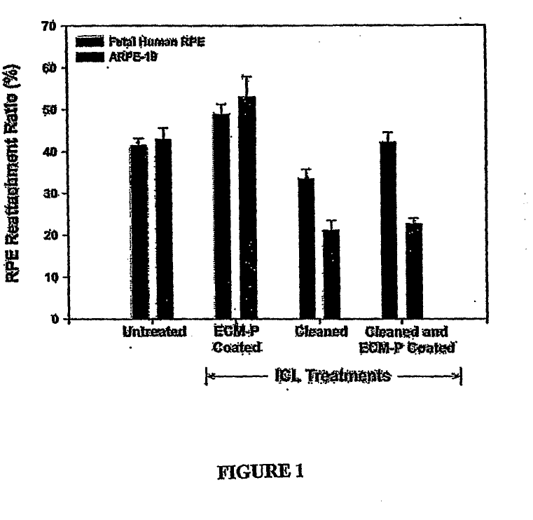

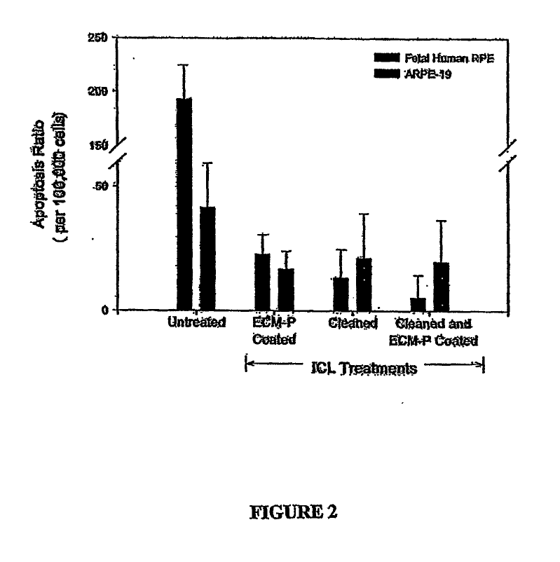

Reattachment ratios of fetal human RPE and ARPE-19 cells on treated and untreated aged (>60 years) human ICL are shown in FIG. 1. Twenty-four hours after plating, the overall reattachment ratios to untreated ICL were comparable for the two cell lines (41.5%±1.7% for fetal human RPE and 42.9%±2.7% for ARPE 19, P>0.05). Coating of the ICL with a mixture of ECM protein increased the reattachment ratios of both cell lines compared with untreated ICL (48.9%±2.3%, P<0.01 for fetal human RPE and 53.0%±4.9%, P<0.01 for ARPE-19). The cleaning procedure significantly decreased the reattachment ratio in both cell lines (33.4%±2.3%, P<0.01 for fetal human RPE and 21.1%±2.3%, P<0.01 for ARPE-19), although the reattachment of ARPE-19 cells was decreased more (P<0.01). Combined cleaning and ECM protein coating restored the re-attachment ratio of fetal human RPE to levels comparable to untreated ICL (42.3%±2.3%, P>0.05), but still remained significantly lower than ECM-protein-coated ICL (P<0.01). In contrast, ECM protein coating after cleaning filed to increase the reattachment ratio of ARPE-19 cells to the level of untreated ICL (22.6%±1.4%, P>0.05).

### Example 8

### Assay for RPE Apoptosis

Twenty-four hours after cells were plated in triplicate wells, the wells were gently washed three times with MEM and fixed with 4% paraformaldehyde for 4 hours. Apoptotic cells were identified using—the-TUNEL method. For this purpose, cells were permeabilized with 0.2% Triton X-100 in 0.2 M sodium citrate solution at 4° C. for 4 minutes. Explants were washed three times with PBS and incubated with a mixture of fluorescein-labeled nucleotides and terminal deoxynucleotidyl transferase (TdT) from calf thymus for 60 minutes. TdT catalyzes the polymerization of labeled nucleotides to free 3′-OH terminals of DNA fragments. DNA breaks were then observed under a fluorescence microscope. For this purpose, explants were carefully remove the wells and flipped over on a coverslip. The total number of apoptotic cells was counted under a fluorescence microscope. The apoptosis ratio on each plating surface was the ratio of apoptotic cells to the total number of attached cells on that surface.

### Example 9

### Apoptosis Ratio

Both cell types demonstrated the highest ratio of apoptosis on untreated ICL (192.7±32.1 per 100,000 cells for fetal human RPE versus 41.5±18.0 per 100,000 cells for ARPE-19 cells, P<0.05; FIG. 2). ARPE-19 cells were significantly more resistant to apoptosis on untreated ICL than were fetal human RPE cells (P<0.01). ECM protein coating lowered the apoptosis ratio significantly and eliminated the difference in apoptosis rates between these two cell populations ((22.7±7.9 per 100,000 fetal human RPE cells and 16.8±7.3 per 100,000 ARPE-19 cells, P>0.05). Cleaning the ICL alone lowered the apoptosis ratios for both cell types, but this was significant only for fetal RPE cells (13.3±11.5 per 100,000 fetal human RPE cells, P<0.05, and 21.1±18.3 per 100,000 ARPE-19 cells, P>0.05). Combined cleaning and ECM protein coating resulted in the lowest apoptosis ratios for fetal human RPE cells (5.3±9.1 per 100,000 fetal human RPE cells, P<0.05). The observed decrease in the apoptosis ratio of ARPE-19 cells on cleaned and ECM-protein-coated ICL was not statistically significant (19.7±17.0 per 100,000 ARPE-19 cells, P>0.05).

### Example 10

### Assay for RPE Proliferation

Twenty-four hours after plating, RPE cell proliferation was stimulated by replacing the medium with MEM supplemented with 15% fetal bovine serum (FBS) and 1 ng/mL recombinant bFGF (Invitrogen-Gibco). The number of cells on each explant was determined with the MTS assay 24 hours after growth stimulation, as described earlier. The proliferation ratio was the ratio of the number of viable and attached cells 24 hours after growth stimulation to the initial number of viable and attached cells on a certain surface.

### Example 11

### Proliferation Ratio

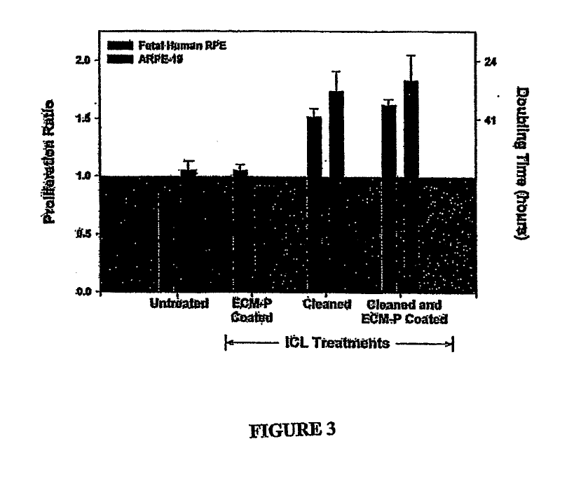

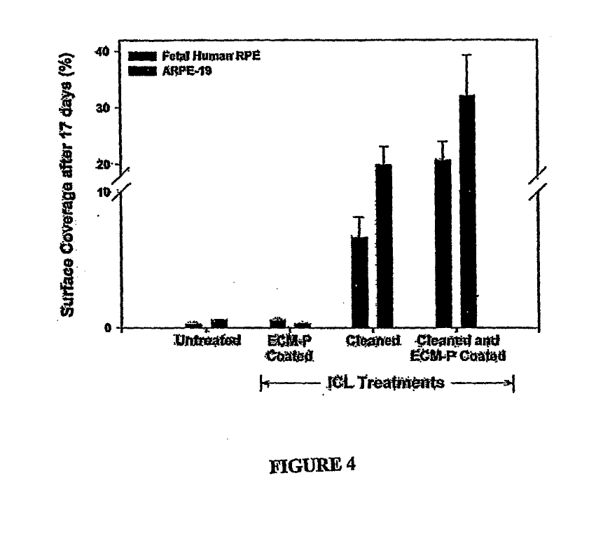

The proliferation ratios of RPE cells 24 hours after growth stimulation on different treatment groups of ICL are shown in FIG. 3. The number of fetal human RPE cells attached to untreated ICL decreased within 24 hours (proliferation ratio=0.87±0.04, P<0.05), whereas the number of ARPE-19 cells remained the same (proliferation ratio=1.05±0.08, P>0.05). The difference between the behavior of these two cell lines was statistically significant (P<0.05). ECM coating prevented loss of fetal RPE (1.05±0.05 for fetal human RPE cells, P<0.01) but had no effect on ARPE-19 (0.91±0.07 for ARPE-19 cells, P>0.05). Cells plated on cleaned ICL increased significantly in number, as evidenced by higher proliferation ratios (1.52±0.07 for fetal human RPE cells and 1.74± for ARPE-19 cells, P<0.01 when compared with untreated ICL). Combined cleaning and ECM coating yielded the highest proliferation ratios in both cell lines (1.62±0.05 for fetal human RPE cells and 1.89±0.22 for ARPE-19 cells, P<0.01 when compared with untreated ICL). These ratios correspond to doubling times of 34.7±2.3 hours for fetal human RPE cells and 28.5±5.1 hours for ARPE-19 cells on cleaned and ECM-protein-coated ICL.

### Example 12

### Assay for RPE Repopulation

In triplicate wells, RPE cells were maintained in 200 μL of MEM containing inert fluorescent beads (Lumafluor, Stony Point, N.Y.) and supplemented with 15% FBS, 100 IU/mL penicillin G, 100 μg/mL streptomycin, 5 μg/mL gentamicin, 2.5 μg/mL amphotericin B, and 1 ng/mL recombinant human bFGF (Invitrogen-Gibco). The culture medium was changed every other day, and cell growth was monitored daily for up to 17 days with an inverted fluorescence microscope (Olympus, Tokyo, Japan) equipped with a 20× long-working-distance objective (numeric aperture [NA]: 0.4, ULWD CDPlano 20PL; Olympus). At the end of the observation period, explants were removed from the wells and mounted upside down on coverslips. Fluorescence microscopy was used to obtain images from 10 representative areas. Total surface coverage, expressed as the percentage of the total surface area, was calculated from the collected images, using image-analysis software (MetaMorph 4.5; Universal Imaging Corporation, Downing-town, Pa.). Results were confirmed with scanning electron microscopy (SEM).

### Example 13

### Ability of RPE Cells to Repopulate ICL

The surface coverage 17 days after initial plating is shown in FIG. 4. RPE cells plated on untreated (0.28%±0.01% for fetal human RPE cells and 0.55%±0.05% for ARPE-19 cells) and ECM-protein-coated ICL (0.55%±0.10% for fetal human RPE cells and 0.30%±0.04% for ARPE-19 cells) failed to survive. RPE cells plated on either cleaned (6.64%±1.51% for fetal human RPE cells and 19.9%+_{—}3.2% for ARPE-19 cells) or cleaned and ECM-protein-coated ICL (20.8%±3.3% for fetal human RPE cells and 32.1%±7.2% for ARPE-19 cells) showed significant surface coverage 17 days after plating. The highest surface coverage was obtained with combined cleaning and ECM protein coating of the ICL.

### Example 14

### Scanning Electron Microscopy

Explants with RPE cells were fixed in modified Karnovsky fixative (2.5% glutaraldehyde and 2% paraformaldehyde in 0.1 M cacodylate buffer [pH 7.4]) at 4° C. overnight. They were then postfixed in 1% osmium tetroxide in 0.16 M cacodylate buffer (pH 7.4) for 1 hour, stained in 1% uranyl acetate in 0.1 M sodium acetate buffer, and dehydrated in a graded series of ethyl alcohol (30%-100%). The samples were then critical point dried (E3000; Polaron, Watford Hertford-shire, UK), mounted on aluminum specimen stubs with carbon-conductive tabs grounded with colloidal silver liquid paint, and sputter coated with 15.0 μm of gold (E5000; Polaron). Samples were examined by SEM (model S.4500 FEG; Hitachi, Tokyo, Japan) at 15 kV accelerating voltage and the images recorded (55 P/N film; Polaroid Corp., Cambridge, Mass.).

### Example 15

### Statistical Analysis

Triplicate wells were used to calculate the average reattachment, apoptosis, and proliferation ratios and the final fate of RPE cells seeded pinto-cacti substrate. Because of the limited number of explants that could be harvested from an eye, typically, SEM studies were performed in duplicate. Data from all experiments were pooled and expressed ±SD. The reattachment, apoptosis, and proliferation surface coverage on different substrates and RPE cell

re analyzed in pairs by the Dunn multiple comparison test.28. A confidence level of P <0.05 was considered to be statistically significant.

### Example 16

### Cellular and Ultrastructural Morphology

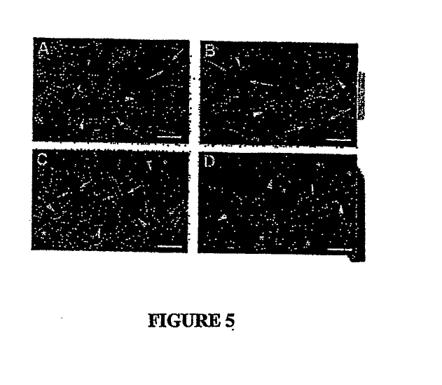

FIG. 5 shows the effect of different treatments on the ultra-structural features of the ICL. Untreated aged ICL mainly consisted of cross-linked collagen fibers arranged in fused bundles that run unidirectionally (FIG. 5A). Delicate interdigitations between collagen fibers were lost, and large pockets of inter-fibrillar spaces were filled with extracellular debris. Globular ECM proteins, which formed aggregates in some locations, were attached along the course of the fibers. ECM protein-coating without cleaning increased the number of globular ECM proteins attached to the fibers (FIG. 5B) with additional smaller clumps of ECM debris on and between cross-linked collagen fibers. Treating the matrix with Triton X-100 and sodium citrate removed the microaggregates and most of the globular ECM proteins, and created empty spaces between bare bundles of collagen fibers (FIG. 5C). Cross-linked collagen bundles partially broke up and a morphologic appearance developed that more closely resembled the native architecture of collagen fibers within Brach's membrane. Cleaning and subsequent coating led to breaking of cross-links, regular spacing of collagen fibers, removal of debris, and homogenous coating with globular structures that may represent clusters of ECM proteins. These clusters were diffusely distributed over the regenerated collagen framework and did not form aggregates as seen on uncleaned ICL (FIG. 5D). Removal of macro-aggregates left empty spaces between collagen fibers.

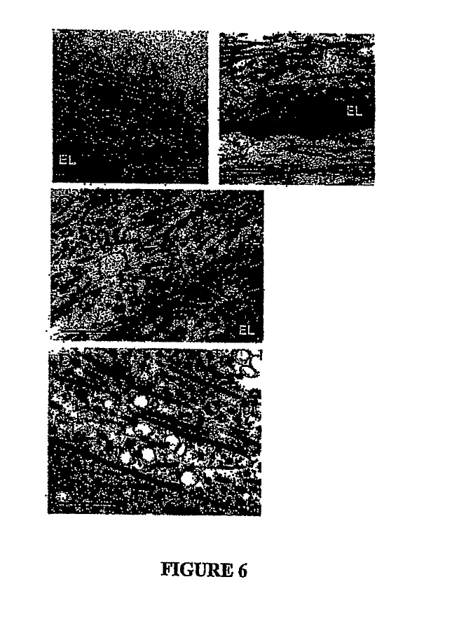

TEM of the untreated aged ICL revealed cross-linked collagen bundles and electron-lucent material that resembled basal linear deposits (FIG. 6A). Occasional electron-dense ECM protein aggregates were noted on the collagen fibers. The addition of ECM proteins without cleaning yielded an increase in the number of electron-dense particles among cross-linked collagen fibers (FIG. 6B). Cleaning the matrix broke the cross-links between collagen fibers, creating gaps in the collagen frame-work (FIG. 6C). After cleaning, an electron dense ring appeared around the electron lucent vesicular debris noted in untreated samples. This probably represents the reconstitution of the proteins within the lipoproteinaceous debris with their hydrophilic surface exposed to the outside, because of the effect of Triton X-100. Electron-dense particles increased considerably among dissociated collagen fibers after the cleaning and coating process. An increase in these anionic binding sites may indicate a shift toward hydrophilicity.

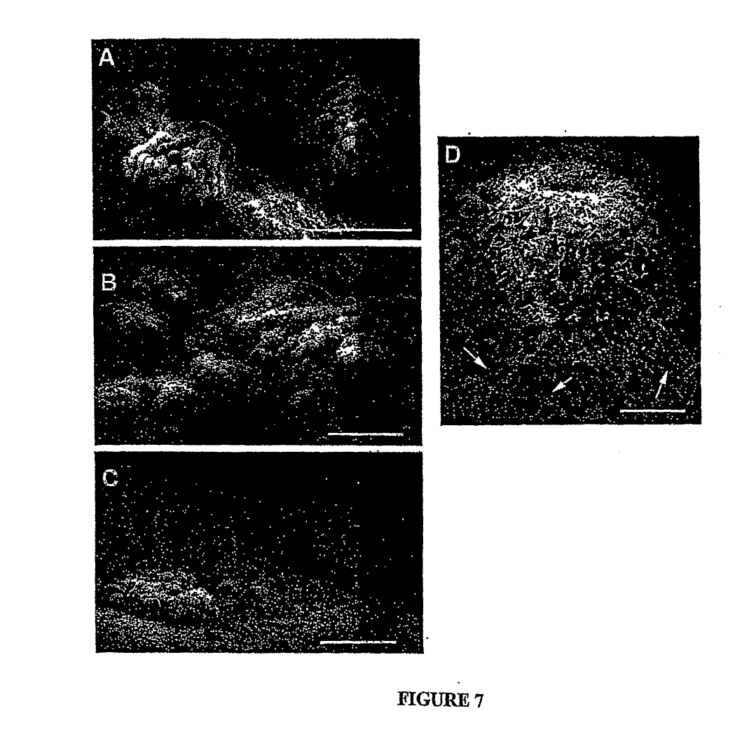

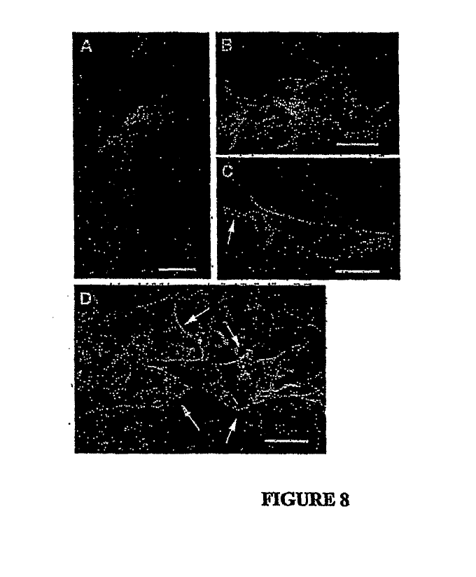

There was no clear difference in the morphology of RPE cell populations seeded onto similar surfaces. RPE cells that attached to untreated ICL and ECM-protein-coated ICL failed to flatten at 24 hours after plating (FIG. 7A). Although ECM protein coating increased the number of attached cells, these cells failed to flatten (FIG. 7B). Cleaning the ICL resulted in the attachment of fewer RPE cells, but the attached RPE cells flattened (FIG. 7C). Combined cleaning and ECM protein coating of the ICL not only resulted in flattening of the attached RPE cells but also in the expression of differentiated RPE cell features, such as apical microvilli (FIG. 7D). On day 17, only rare cellular debris was noted on untreated and ECM-protein-coated ICL (FIG. 8A, 8B). A few RPE cells plated onto the cleaned surface flattened and developed long extensions (FIG. 8C), but cleaned and ECM-protein-coated ICL was markedly different, with many patches of the ICL covered by a RPE monolayer (FIG. 8D).

### Example 17

### Effects on RPE Behavior of Cleaning and Recoating Bruch's Membrane

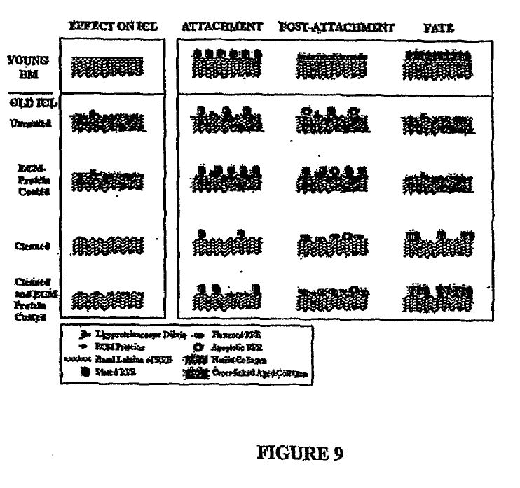

FIG. 9 demonstrates the effects of cleaning and resurfacing the ICL on RPE cell attachment, apoptosis, and proliferation. Young Bruch's membrane contains regularly spaced collagen fibers with no significant cross-linking or accumulation of extracellular deposits. ECM ligands, such as laminin fibronectin and vitronectin, are present (FIG. 9, column 1, row 1). Changes develop within the ICL as a function of age, including collagen cross-linking and the accumulation of ECM deposits (FIG. 9, column 1, row 2). Coating the ICL without cleaning increases the number of receptors available on the inner aspects of the ICL but collagen cross-links and lipoproteinaceous debris are still present (FIG. 9, column 1, row 3). Cleaning old ICL reverses the collagen cross-linking and removes the ECM deposits, but also removes most of the ECM ligands necessary for cell attachment (FIG. 9, column 1, row 4). Coating of cleaned ICL refurbishes Bruch's membrane by removing the deposits, removing the cross-links, and then providing again a normal complement of ECM ligands for cell attachment (FIG. 9, column 1, row 5).

In FIG. 9, columns 2 to 4 show the effects of these ultrastructural changes on RPE attachment, apoptosis, and proliferation. The RPE attachment and proliferation rates are high, and apoptosis is low on young ICL (FIG. 9, row 1). Age-related changes in the ICL of Brunch's membrane adversely affect RPE attachment and proliferation rates and increase the RPE apoptosis rate (FIG. 9, row 2). Simple ECM protein coating alone is not sufficient to restore in the integrity of Bruch's membrane for RPE behavior. Coating alone significantly increases RPE attachment to the abnormal surface, probably by simply supplying the necessary ECM ligands. Despite the decreased apoptosis rate, the proliferation rate remains low when only the surface is coated (FIG. 9, row 3), suggesting that different cell-surface interactions control each of these functions. Cleaning alone lowers the attachment rate further, probably by removing some normal ECM ligands present within the ICL, but the attached cells flatten and undergo proliferation, unlike RPE cell on ECM-protein-coated ICL (FIG. 9, row 4).

Cleaning followed by ICL resurfacing appears to reverse the age-related changes partially and maximize the surface repopulation of the ICL (FIG. 9, row 5). Cleaning and ECM protein coating restores RPE cell attachment, subsequent cell spreading and proliferation and decreases the apoptosis rate. Electron microscopy reveals that surface cleaning eliminates the collagen cross-links and removes ECM deposited. At the same time, the process of removing the debris and regenerating the collagen framework probably vacates binding sites of ECM proteins on collagen fibers, onto which newly added ECM proteins can polymerize to create a structure that is closer to the native ultrastructural architecture.

The foregoing examples are illustrative of the present invention and are not to be construed as limiting thereof.

What is claimed is:

1. A method of reversing age-related changes in extracellular matrix components, comprising
i. applying to a membrane comprising the extracellular matrix components with a composition comprising
a. a non-ionic detergent, a biodetergent, or a lipid-degrading enzyme; and
b. a weak acid; and
ii. coating said membrane with a mixture of extracellular matrix protein.

2. The method of claim 1, where the extracellular matrix component is collagen

3. The method of claim 1 or claim 2, further comprising an intermediate step of applying an agent capable of breaking the collagen cross-links in the inner matrix layer.

4. The method of claim 3, where the intermediate step of applying an agent capable of breaking the collagen cross-links in the inner matrix layer is carried out using N-phenyl thiazolium bromide.

5. The method of claims 1-4, where the mixture of extracellular matrix protein is a composition comprising laminin, fibronectin, and vitronectin.

6. The method of claim 5, where said mixture of extracellular matrix protein contains from about 300 to about 350 micrograms/ml of laminin; from about 220 to about 280 micrograms/ml of fibronectin; and from about 30 to about 50 micrograms/ml of vitronectin.

7. The method of claim 6, where said extracellular matrix protein composition contains from about 300 to about 400 micrograms/ml of laminin; from about 200 to about 300 micrograms/ml of fibronectin; and from about 30 to about 40 micrograms/ml of vitronectin.

8. The method of any of claims 1-7, where the method is carried out using a non-ionic detergent.

9. The method of claim 8, where the non-ionic detergent is Triton-X.

10. The method of any of claims 1-7, where the method is carried out using a biodetergent.

11. The method of any of claims 1-7, where the method is carried out using a lipid-degrading enzyme.

12. The method of any of claims 1-11, where the weak acid is an organic acid whose pKa is between about 3 and about 6.

13. The method of any of claims 1-11, where the weak acid is an organic acid whose pKa is between about 3 and about 5.

14. The method of any of claims 1-11, where the weak acid is sodium citrate.

15. The method of any of claims 1-14, where the matrix component is collagen in the basement membrane of the eye.

16. The method of any of claims 1-14, where the matrix component is collagen in the skin.

17. The method of any of claims 1-14, where the component is collagen in an internal organ.

18. A method of removing accumulated beta-amyloid protein, comprising
i. contacting the beta-amyloid protein with a non-ionic detergent and a weak acid;
ii. optionally, applying to the beta-amyloid protein or between the beta-amyloid protein and other ECM proteins a second composition comprising N-phenyl thiazolium bromide; and
iii. treating said beta-amyloid protein with extracellular matrix proteins.

19. A method of treating age-related macular degeneration, comprising
i. contacting the inner collagen layer of Bruch's membrane with a non-ionic detergent and a weak acid;
ii. treating said inner collagen layer with extracellular matrix proteins.

20. The method of claim 14, further comprising applying N-phenyl thiazolium bromide to the collagen cross-links in the inner collagen layer.

21. The method of claims 18-20, where the detergent is Triton-X.

22. The method of claims 18-20, where the weak acid is an organic acid whose pKa is between about 3 and about 6.

23. A method of repairing age-related damage in Bruch's membrane, comprising,
i. applying to the inner collagen layer of Bruch's membrane a non-ionic detergent and a weak acid; and
ii. treating said inner collagen layer with extracellular matrix proteins.

24. The method of claim 23, further comprising applying a second composition comprising N-phenyl thiazolium bromide.

25. A method of preventing open-angle glaucoma, comprising
i. applying to the deteriorated matrix of the trabecular meshwork a non-ionic detergent and a weak acid; and
ii. treating said trabecular meshwork with extracellular matrix proteins.

26. The method of claim 25, further comprising applying a second composition comprising N-phenyl thiazolium bromide.

27. A method of treating skin wrinkling due to age or sun damage, comprising
i. applying with a non-ionic detergent and a weak acid to the skin; and
ii. coating the skin with extracellular matrix proteins.

28. The method of claim 27, further comprising applying a second composition comprising N-phenyl thiazolium bromide.

29. The method of claims 18-28, where the extracellular matrix proteins are laminin, fibronectin, and vitronectin.

30. A method of treating AMD comprising
the procedure of claim 1 or claim 3; followed by
the addition of retinal pigment epithelial (“RPE”) cells.

31. The method of claim 30, where the RPE cells are fetal RPE cells.

32. A method of reversing age-related changes in collagen protein, comprising
i. applying to a membrane comprising said collagen protein a composition comprising
a. a non-ionic detergent; and
b. a weak acid;
ii. coating said membrane with extracellular matrix protein
iii. adding retinal pigment epithelial (RPE) cells.

33. The method of any of claims 18-32, where the detergent is a biodetergent.

34. The method of claim 26, where the biodetergent is a fluorinated surfactant.

35. The method of claim 26, where the biodetergent is replaced by a lipid-degrading enzyme.
